(19) United States
(12) Patent Application Publication
(10) Pub. No.: US 2014/0277108 A1
(43) Pub. Date: Sep. 18, 2014

(54) SURGICAL INSTRUMENT FOR GRASPING MULTIPLE LOCATIONS AND METHODS OF USE

(71) Applicant: THE OHIO STATE UNIVERSITY, Columbus, OH (US)

(72) Inventor: David B. Renton, Powell, OH (US)

(73) Assignee: THE OHIO STATE UNIVERSITY, Columbus, OH (US)

(21) Appl. No.: 13/795,415

(22) Filed: Mar. 12, 2013

(51) Int. Cl. A61B 17/00; A61B 17/29

(52) U.S. Cl. CPC A61B 17/00234; A61B 17/2909
USPC 606/206; 606/1; 606/205

(57) ABSTRACT

According to various implementations, a surgical instrument is provided that has two graspers for remotely grasping an organ or tissue in two different locations. The surgeon can manipulate the organ or tissue using the instrument with one hand while using his or her other hand to perform a surgical procedure. Using one instrument also reduces the number of incisions required. This instrument is especially useful in laproscopic procedures and other minimally invasive surgeries.

### BACKGROUND

Implementations of the present invention relate to surgical instruments and methods of grasping multiple locations on an organ or tissue during surgical procedures.

During a surgical procedure, a surgeon may need to manipulate an organ or tissue. Surgeons may utilize a grasper to access or manipulate the organ or tissue, such as the instrument described in U.S. Pat. No. 5,402,342, which is herein incorporated by reference. However, known grasping instruments only provide one grasper per instrument. Therefore, surgeons have had to use two or more instruments to grasp the organ or tissue in two different locations to manipulate the organ or tissue with sufficient dexterity. One instrument may be manipulated by the surgeon, but the other instrument is typically manipulated by an assistant while the surgeon uses his or her other hand to perform a surgical procedure. One drawback to using two or more instruments is that each instrument requires its own incision. Another drawback is that the surgeon has to communicate with the assistant to coordinate the movements of each instrument in manipulating the organ or tissue.

Accordingly, an improved surgical instrument and method are needed to provide improved manipulation of organs or tissues within the body during surgery and to reduce the number of incisions required.

### BRIEF SUMMARY

According to various implementations, a surgical instrument is provided that has two graspers for remotely grasping an organ or tissue in two different locations. The surgeon can manipulate the organ or tissue using the instrument with one hand while using his or her other hand to perform a surgical procedure. Using one instrument also reduces the number of incisions required. This instrument is especially useful in laproscopic procedures and other minimally invasive surgeries.

In particular, a surgical instrument according to various implementations includes a handle configured for being held in one hand, an elongated portion extending from the handle substantially along an axis, a first pair of cooperating end effectors operatively disposed adjacent a distal end of the elongated portion, and a second pair of cooperating end effectors operatively disposed adjacent the distal end of the elongated portion. The handle includes an extension mechanism and a linear extension mechanism. The extension mechanism is configured for extending distal ends of the first pair of cooperating end effectors outwardly from the distal end of the elongated portion and laterally relative to the axis. The linear extension mechanism is configured for urging distal ends of the second pair of cooperating end effectors outwardly from the distal end of the elongated portion and substantially parallel to the axis. The instrument may also include a locking mechanism configured for preventing movement of the distal ends of the first cooperating pair of end effectors relative to each other.

In one implementation in which the end effectors are graspers, the second pair of graspers may include first and second leaflets that are made of a spring-like material. The first and second leaflets may include distal ends that are biased away from each other and proximate ends opposite the distal ends that are held in a fixed position relative to each other. In addition, each leaflet may define a serrated surface that substantially mates with the serrated surface of the opposing leaflet when the leaflets are compressed toward each other.

The linear extension mechanism may include a push rod that has a proximate end disposed adjacent the handle and a distal end disposed adjacent the proximate ends of the leaflets. The push rod is slidable in a first direction that is substantially parallel to the axis to urge the distal ends of the leaflets outwardly from the distal end of the elongated portion. The push rod is slidable in a second, opposite direction to urge the proximate ends of the leaflets toward the distal end of the elongated portion. The instrument may also include a second locking mechanism configured for preventing axial movement of the push rod.

Alternatively, the linear extension mechanism may include a threaded rod that has a proximate end disposed adjacent the handle and a distal end disposed adjacent the distal end of the elongated portion. The proximate end of the threaded rod is configured for rotation in a first direction about its axis to move a distal end of the threaded rod outwardly from the distal end of the elongated portion. The rod is rotatable in a second, opposite direction about its axis to move the distal end of the threaded rod toward the distal end of the elongated portion.

The extension mechanism may be configured for remotely extending distal ends of the first pair of graspers up to around 7 cm from distal ends of the second pair of graspers. For example, in one implementation, the extension mechanism includes an axially movable member disposed adjacent the handle and a proximate end of the elongated portion, which is an outer elongated portion. The extension mechanism also includes a fixed, inner elongated portion mounted coaxially within at least a portion of the outer elongated portion and an extension member comprising a shape memory alloy. The extension member has a distal end disposed adjacent the first pair of graspers and a proximate end disposed adjacent a distal end of the inner elongated portion. The shape memory alloy is bent at an angle greater than 0 degrees when unstressed and is substantially coaxial with the inner elongated portion when stressed. The axially movable member is configured for being axially translated in a first axial direction to move the outer elongated portion relative to the inner elongated portion, which stresses the extension member such that the extension member is substantially coaxial with the inner elongated portion. In addition, the axially movable member is configured for being axially translated in a second, opposite axial direction, which releases stress on the extension member such that the extension member extends laterally from an axis of the inner elongated portion. In one implementation, the axially movable member is a slidable barrel assembly. In another implementation, the axially movable member is a drive screw assembly.

A method of using a surgical instrument according to one implementation includes: (1) providing a surgical instrument that includes a handle, an elongated portion extending from the handle along an axis, and first and second pairs of graspers disposed adjacent a distal end of the elongated portion; (2) guiding at least a portion of the elongated portion of the surgical instrument through a small incision in a patient's body; (3) remotely extending the first and second pairs of graspers away from the distal end of the elongated portion toward a tissue or organ in the patient's body; (4) remotely positioning the first pair of graspers into a first position and the second pair of graspers into a second position; (5) grasping the tissue or organ in a first location adjacent the first position; (6) grasping the tissue or organ in a second location adjacent the second position, the first location being spaced apart from the second location; and (7) after grasping the tissue or organ with the first and second pairs of graspers, moving the handle of the surgical instrument with one hand to manipulate the tissue or organ. The method may further include locking the first pair of graspers into the first position or the second pair of graspers into the second position, or both.

### BRIEF DESCRIPTION OF THE DRAWINGS

FIG. 1 illustrates a side view of a surgical instrument according to one implementation.

FIG. 2 illustrates a partial side view of a handle of the surgical instrument shown in FIG. 1.

FIG. 3 illustrates a cut-away side view of the surgical instrument shown in FIG. 1.

FIG. 4 illustrates a partial perspective view of the surgical instrument shown in FIG. 1.

FIG. 5 illustrates a side view of the surgical instrument according to an alternative implementation.

FIG. 6 illustrates a partial cut-away side view of the surgical instrument shown in FIG. 5.

FIG. 7 illustrates a partial side view of a handle of a surgical instrument according to an alternative implementation.

FIG. 8 illustrates a method of using a surgical instrument.

### DETAILED DESCRIPTION

According to various implementations, a surgical instrument is provided that has two graspers for remotely grasping an organ or tissue in two different locations. The surgeon can manipulate the organ or tissue using the instrument with one hand while using his or her other hand to perform a surgical procedure. Using one instrument also reduces the number of incisions required. This instrument is especially useful in laproscopic procedures and other minimally invasive surgeries.

As shown in FIG. 1, the surgical instrument 10 includes a handle 11 configured for being held in one hand, an elongated portion 12 that extends from the handle 11 substantially along an axis A-A, a first pair of cooperating end effectors 13, and a second pair of cooperating end effectors 15. The first pair of end effectors 13 and the second pair of end effectors 15 are operatively disposed adjacent a distal end 14 of the elongated portion 12.

The handle 11 includes an extension mechanism 16 and a linear extension mechanism 18. The extension mechanism 16 is configured for extending distal ends 17 of the first pair of cooperating end effectors 13 outwardly from the distal end 14 of the elongated portion 12 and laterally relative to the axis A-A. The linear extension mechanism 18 is configured for urging distal ends 19 of the second pair of cooperating end effectors 15 outwardly from the distal end 14 of the elongated portion 12 and substantially parallel to the axis A-A. The end effectors 13, 15 may be graspers, scissors, dissectors, or clamps.

In one implementation in which the second pair of end effectors 15 are a pair of graspers, the second pair of graspers 15 may include a first leaflet 20 and a second leaflet 21 that are made of a spring-like material, such as tungsten. The first and second leaflets 20, 21 may include distal ends 19 that are biased away from each other and proximate ends 22 opposite the distal ends 19 that are held in a fixed position relative to each other. For example, in one implementation in which the leaflets 20, 21 are formed from one piece of material, the proximate ends 22 of each leaflet 20, 21 may be the fold in the material. In another implementation in which the leaflets 20, 21 comprise two separate pieces of material, the proximate ends 22 may be welded together or joined by other suitable means. In addition, each leaflet 20, 21 may define a serrated surface 23 that substantially mates with the serrated surface 23 of the opposing leaflet when the leaflets 20, 21 are compressed toward each other.

The linear extension mechanism 18 may include a push rod 28 that has a proximate end 24 disposed adjacent the handle 11 and a distal end 25 disposed adjacent the proximate ends 22 of the leaflets 20, 21. The proximate end 24 of the push rod 28 is slidable in a first direction that is substantially parallel to the axis A-A, such as toward the handle 11, to urge the distal ends 19 of the leaflets 20, 21 outwardly from the distal end 14 of the elongated portion 12. The proximate end 24 is slidable in a second, opposite direction, such as away from the handle 11, to urge the proximate ends 22 of the leaflets 20, 21 toward the distal end 14 of the elongated portion 12. When the proximate ends 22 of the leaflets 20, 21 pass through the distal end 14 of the elongated portion 12, the distal ends 19 of the leaflets 20, 21 are urged toward each other, allowing them to grasp an organ or tissue. In addition, the proximate end 24 of the push rod 28 may be rotated about its axis to remotely rotate the leaflets 20, 21 into position for grasping the organ or tissue.

The instrument 10 may also include a locking mechanism configured for preventing axial movement of the push rod, such as the locking mechanism illustrated in FIG. 2. In particular, the locking mechanism includes at least one notch 26 defined in the surface of the push rod 28 axially inwardly from the proximate end 24 of the push rod 28 and a pawl 27 that extends through the handle 11 for selectively engaging the notch 26. The handle 11 defines an opening 29 through which the pawl 27 slides. In another implementation (not shown), the locking mechanism may include a leaf spring disposed on the surface of the push rod 28 that is configured for selectively engaging a depression defined in a channel of the handle 11 through which the push rod 28 slides.

The extension mechanism 16 may be configured for remotely extending distal ends 17 of the first pair of graspers 13 outwardly and laterally relative to the axis A-A, such as up to about 90 degrees from the axis A-A. This extension may allow the distal end 17 of the first pair of graspers 13 to be spaced apart from the distal end 19 of the second pair of graspers 15 up to around 7 cm.

FIG. 3 illustrates an exemplary implementation of the extension mechanism 16. In particular, the extension mechanism 16 includes a slidable barrel assembly 61 disposed adjacent the handle 11 and a proximate end 30 of the elongated portion 12. The elongated portion 12 includes an outer elongated portion 64. The extension mechanism 16 also includes a fixed, inner elongated portion 63 mounted coaxially within at least a portion of the outer elongated portion 64 and an extension member 31 comprising a shape memory alloy. The extension member 31 has a distal end 32 disposed adjacent the first pair of graspers 13 and a proximate end 33 disposed adjacent a distal end 63 of the inner elongated portion 63.

The shape memory alloy is bent at an angle greater than 0 degrees when unstressed and is substantially coaxial with the inner elongated portion 63 when stressed. The slidable barrel assembly 61 is configured for being axially translated in a first axial direction to move the outer elongated portion 64 relative to the inner elongated portion 63, which stresses the extension member 31 such that the extension member 31 is substantially coaxial with the inner elongated portion 63. In addition, the slidable barrel assembly 61 is configured for being axially translated in a second, opposite axial direction, which releases stress on the extension member 31 such that the extension member 31 extends laterally from an axis of the inner elongated portion 63.

As shown in FIGS. 3 and 4, the outer elongated portion 64 comprises a first channel 43 and a second channel 44. The inner elongated portion 63 extends through the first channel 43, and the push rod 28 extends through the second channel 44. As shown, the first channel 43 is below the second channel 44, but in other implementations, the second channel 44 may be disposed below the first channel 43 or side by side. According to one implementation, the outer elongated portion 64 is around 35 cm long and around 5 mm in diameter.

The handle 11 further comprises an axially rotatable collar 40 that is operatively engaged with a proximate end 62 of the inner elongated portion 63 such that rotation of the collar 40 about an axis of the inner elongated portion 63 rotates the inner elongated portion 63 and the extension member 31 about the axis of the inner elongated portion 63. As shown in FIG. 1, a portion of the collar 40 extends through the handle 11 to allow the operator to rotate it about the axis of the inner elongated portion 63. When the collar 40 is rotated in a first direction, the inner elongated portion 63 is rotated about its axis in the first direction. Similarly, when the collar 40 is rotated in a second direction, the inner elongated portion 63 is rotated about its axis in the second direction. U.S. Pat. No. 5,403,342 describes various implementations of the operable connection between the inner elongated portion 63 and the rotatable collar 40.

A cable assembly is configured for remotely opening and closing the first pair of graspers 13 in response to moving a pivoting portion 35 of the handle 11 relative to a fixed portion 36 of the handle 11. One implementation of the cable assembly is shown in FIGS. 3 and 4. In particular, as shown in FIG. 4, the proximate ends 22 of the first pair of graspers 13 define elongated slots 122 and are rotatably fixed together about fixed pin 125, which is disposed adjacent distal ends of the elongated slots 122. A pivot pin 123 extends through the slots. A cable 124 extends through the extension member 31 and the inner elongated portion 63 between the pivot pin 123 and the pivoting portion 35 of the handle 11. When an upper portion of the pivoting portion 35 of the handle 11 is moved away from the fixed portion 36, the cable 124 urges the pivot pin 123 toward proximate ends of the elongated slots 122, which urges the distal ends 17 of the first pair of graspers 13 toward each other. When the upper portion of the pivoting portion 35 of the handle 11 is moved toward the fixed portion 36, the cable 124 urges the pivot pin 123 toward distal ends of the elongated slots 122, which urges distal ends 17 of the first pair of graspers 13 away from each other. Other implementations of mechanisms for remotely operating the first pair of graspers may be suitable for this surgical instrument, such as those mechanisms described in U.S. Pat. No. 5,403,342.

The instrument 10 also includes a locking mechanism configured for preventing movement of the distal ends 17 of the first cooperating pair of end effectors 13 relative to each other. For example, as shown in FIG. 3, the locking mechanism includes a pawl 37 and a ratchet 38. The ratchet 38 is defined on the pivoting portion 35 of the handle 11. The pawl 37 is disposed adjacent the handle 11 and is configured for selectively engaging the ratchet 38 and preventing movement of the pivoting portion 35 in at least one direction. In particular, the pawl 37 extends into the fixed portion 36 of the handle 11 from a slidable tab 50. The fixed portion 36 of the handle 11 defines an elongated slot 34 through which the pawl 38 can travel when the tab 50 is slide along the handle 11. When the pawl 38 is slid into position B, the pawl 38 cannot engage the ratchet 37, which allows the pivoting portion 35 of the handle 11 to be operated without restriction. However, if the pawl 38 is slid toward the ratchet 37, the pawl 38 engages the ratchet 37 when the pivoting portion 35 of the handle 11 is moved toward the fixed portion 36 of the handle. Other suitable locking mechanisms may be described in U.S. Pat. No. 5,403,342.

FIGS. 5 and 6 illustrate an alternative embodiment of the extension mechanism that includes a drive screw assembly 49 to axially translate the outer elongated portion 64 relative to the inner elongated portion 63. In particular, the drive screw assembly 49 includes an outer barrel 45 that defines an axial bore 41 and a threaded area 42 on the surface of the axial bore 41. The assembly 49 also includes a drive screw 46 that defines a threaded area 48 on an outer surface of the drive screw 46. The threaded area 48 is configured for engaging the threaded area 42 in the outer barrel 45. The outer barrel 45 is disposed adjacent a distal end 47 of the handle and is rotatable about axis A-A but is held in a fixed axial position between the distal end 47 of the handle 11 and flange 39. A distal end of the drive screw 46 is axially engaged with a proximate end 66 of the outer elongated portion 65. When the outer barrel 45 is rotated in a first direction about the axis A-A, the drive screw 46 is moved axially through the threaded areas 42, 48, which axially moves the outer elongated portion 65 relative to the inner elongated portion 63 and stresses the extension member 31. When the outer barrel 45 is rotated in a second opposite direction, the drive screw 46 is translated axially in an opposite axial direction, which axially translates the outer elongated portion 65 relative to the inner elongated portion 63 and releases stress on the extension member 31. U.S. Pat. No. 5,403,342 describes various other implementations of axial movable members and other suitable extension mechanisms for extending the first pair of graspers 13 outwardly and laterally relative to axis A-A.

FIG. 7 illustrates an alternative implementation of the linear extension mechanism. In this implementation, the linear extension mechanism 58 may include a threaded rod 50 that has a proximate end 51 disposed adjacent the handle 11 and a threaded portion 52 defined on the surface of the rod 50 axially inwardly of the proximate end 51. The handle 59 defines a threaded channel 53 that is configured for operatively mating with the threaded portion 52 of the threaded rod 50. The proximate end 51 of the threaded rod 50 is rotatable in a first direction about its axis to move the distal end of the threaded rod 50 outwardly from the distal end 14 of the elongated portion 12. The proximate end 51 of the threaded rod 50 is rotatable in a second, opposite direction about its axis to move the distal end of the threaded rod 50 toward the distal end 14 of the elongated portion 12.

FIG. 8 illustrates a method of using a surgical instrument, such as the surgical instrument 10 described above in relation to FIGS. 1 through 7. In step 101, a surgical instrument is provided. The surgical instrument includes a handle, an elongated portion extending from the handle along an axis, and first and second pairs of graspers disposed adjacent a distal end of the elongated portion. In step 102, at least a portion of the elongated portion of the surgical instrument is guided through a small incision in a patient's body. In one implementation, a trochar may be inserted through the small incision, and the distal end of the elongated portion may be guided through it. In step 103, the first and second pairs of graspers are remotely extended away from the distal end of the elongated portion toward a tissue or organ in the patient's body. In step 104, the first pair of graspers is remotely positioned into a first position and the second pair of graspers is remotely positioned into a second position. In step 105, the tissue or organ is grasped in a first location adjacent the first position. In step 106, the tissue or organ is grasped in a second location adjacent the second position. The first location is spaced apart from the second location. After grasping the tissue or organ with the first and second pairs of graspers, the handle of the surgical instrument is moved with one hand to manipulate the tissue or organ, as shown in step 107. According to a further implementation, the method may further include locking the first pair of graspers into the first position or the second pair of graspers into the second position, or both.

The described methods, systems, and apparatus should not be construed as limiting in any way. Instead, the present disclosure is directed toward all novel and nonobvious features and aspects of the various disclosed implementations, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

1. A surgical instrument comprising:
a handle configured for being held in one hand;
an elongated portion extending from the handle substantially along an axis;
a first pair of cooperating end effectors operatively disposed adjacent a distal end of the elongated portion; and
a second pair of cooperating end effectors operatively disposed adjacent the distal end of the elongated portion,
wherein the handle comprises:
an extension mechanism configured for extending distal ends of the first pair of cooperating end effectors outwardly from the distal end of the elongated portion and laterally relative to the axis, and
a linear extension mechanism configured for urging distal ends of the second pair of cooperating end effectors outwardly from the distal end of the elongated portion and substantially parallel to the axis.

2. The surgical instrument of claim 1, wherein the first and second pairs of cooperating end effectors are graspers.

3. The surgical instrument of claim 2, wherein the second pair of graspers comprises first and second leaflets, the leaflets comprising distal ends that are biased away from each other and proximate ends opposite the distal ends that are held in a fixed position relative to each other.

4. The surgical instrument of claim 3, wherein each leaflet defines a serrated surface that substantially mates with the serrated surface of the opposing leaflet when the leaflets are compressed toward each other.

5. The surgical instrument of claim 3, wherein the leaflets comprise a spring-like material.

6. The surgical instrument of claim 3, wherein the linear extension mechanism comprises a push rod, the push rod comprising a proximate end disposed adjacent the handle and a distal end disposed adjacent the proximate ends of the leaflets, wherein the push rod is slidable in a first direction that is substantially parallel to the axis to urge the distal ends of the leaflets outwardly from the distal end of the elongated portion and is slidable in a second, opposite direction to urge the proximate ends of the leaflets toward the distal end of the elongated portion.

7. The surgical instrument of claim 6, wherein the handle further comprises a locking mechanism configured for preventing axial movement of the push rod.

8. The surgical instrument of claim 7, wherein the locking mechanism comprises at least one notch defined on a surface of the push rod adjacent the handle and a pawl configured for selectively engaging the notch and preventing axial movement of the push rod.

9. The surgical instrument of claim 3, wherein the linear extension mechanism comprises a threaded rod, the threaded rod having a proximate end disposed adjacent the handle and a distal end disposed adjacent the distal end of the elongated portion, the proximate end of the threaded rod being configured for rotation in a first direction about its axis to move a distal end of the threaded rod outwardly from the distal end of the elongated portion and for rotation in a second, opposite direction about its axis to move the distal end of the threaded rod toward the distal end of the elongated portion.

10. The surgical instrument of claim 2, wherein the extension mechanism is configured for remotely extending the distal ends of the first pair of graspers up to around 7 cm from the distal ends of the second pair of graspers.

11. The surgical instrument of claim 10, wherein the elongated portion is an outer elongated portion and the extension mechanism comprises:
an axially movable member disposed adjacent the handle and a proximate end of the outer elongated portion;
a fixed, inner elongated portion mounted coaxially within at least a portion of the outer elongated portion; and
an extension member comprising a shape memory alloy and having a distal end disposed adjacent the first pair of graspers and a proximate end disposed adjacent a distal end of the inner elongated portion, the shape memory alloy being bent at an angle greater than 0 degrees when unstressed and being substantially coaxial with the inner elongated portion when stressed;
wherein the axially movable member is configured for being axially translated in a first axial direction to move the outer elongated portion relative to the inner elongated portion, the axial movement of the outer elongated portion in the first axial direction being configured to stress the extension member such that the extension member is substantially coaxial with the inner elongated portion, and
wherein the axially movable member is configured for being axially translated in a second, opposite axial direction, the axial movement of the outer elongated portion in the second axial direction being configured to release stress on the extension member such that the extension member extends laterally from an axis of the inner elongated portion.

12. The surgical instrument of claim 11, wherein the axially movable member is a slidable barrel assembly.

13. The surgical instrument of claim 11, wherein the axially movable member is a drive screw assembly.

14. The surgical instrument of claim 11, wherein the handle further comprises an axially rotatable collar, the collar operatively engaged with a proximate end of the inner elongated portion such that rotation of the collar rotates the extension member about its axis.

15. The surgical instrument of claim 11, further comprising a cable assembly comprising a cable that extends through the inner elongated portion and the extension member and between a pivoting portion of the handle and proximate ends of the first pair of graspers, wherein the cable assembly is configured for opening and closing the first pair of graspers in response to moving the pivoting portion relative to a fixed portion of the handle.

16. The surgical instrument of claim 11, wherein:
the outer elongated portion defines a first channel and a second channel,
the inner elongated portion is disposed within the first channel, and
the linear extension mechanism comprises a second extension member, the second extension member extending through the second channel between the handle and the second cooperating pair of end effectors.

17. The surgical instrument of claim 1, further comprising a locking mechanism configured for preventing movement of the distal ends of the first cooperating pair of end effectors relative to each other.

18. The surgical instrument of claim 17, wherein the locking mechanism comprises a pawl and a ratchet, the ratchet being defined on a pivoting portion of the handle and the pawl being disposed adjacent the handle, wherein the pawl is configured for selectively engaging the ratchet and preventing movement of the pivoting portion in at least one direction.

19. The surgical instrument of claim 1, wherein the extension mechanism is configured for extending the distal ends of the first pair of cooperating end effectors outwardly from the distal end of the elongated portion and up to about 60 degrees relative to the axis.

20. The surgical instrument of claim 1, wherein the extension mechanism is configured for extending the distal ends of the first pair of cooperating end effectors outwardly from the distal end of the elongated portion and up to about 90 degrees relative to the axis.

21. A method of using a surgical instrument, comprising:
providing a surgical instrument, the surgical instrument comprising a handle, an elongated portion extending from the handle along an axis, and first and second pairs of graspers disposed adjacent a distal end of the elongated portion;
guiding at least a portion of the elongated portion of the surgical instrument through a small incision in a patient's body;
remotely extending the first and second pairs of graspers away from the distal end of the elongated portion toward a tissue or organ in the patient's body;
remotely positioning the first pair of graspers into a first position and the second pair of graspers into a second position;
grasping the tissue or organ in a first location adjacent the first position;
grasping the tissue or organ in a second location adjacent the second position, the first location being spaced apart from the second location; and
after grasping the tissue or organ with the first and second pairs of graspers, moving the handle of the surgical instrument with one hand to manipulate the tissue or organ.

22. The method of claim 21, wherein remotely extending the first pair of graspers comprises rotating a drive assembly disposed adjacent the handle in a first direction.

23. The method of claim 22, wherein the handle further comprises a rotatable collar configured for rotating the first pair of graspers, and remotely positioning the first pair of graspers comprises rotating the collar.

24. The method of claim 23, wherein remotely positioning the first pair of graspers further comprises rotating the drive assembly in the first direction or in a second, opposite direction, wherein rotation in the second direction urges the first pair of graspers toward the distal end of the elongated portion.

25. The method of claim 21, wherein remotely extending the first pair of graspers comprises sliding a slidable barrel assembly disposed adjacent the handle in a first direction.

26. The method of claim 21, wherein remotely extending the second pair of graspers comprises pushing a proximate end of a push rod toward the handle.

27. The method of claim 21, wherein grasping the tissue or organ in the second location comprises remotely retracting the second pair of graspers toward the distal end of the elongated portion such that distal ends of the second pair of graspers grasp the second location.

28. The method of claim 27, wherein remotely retracting the second pair of graspers comprises pulling the proximate end of the push rod away from the handle.

29. The method of claim 21, further comprising locking the second pair of graspers into the second position.

30. The method of claim 29, wherein the push rod defines at least one notch and the handle comprises at least one pawl, and locking the second pair of graspers into the second position comprises engaging the pawl into the notch to prevent axial movement of the push rod.

31. The method of claim 21, wherein remotely operating the first pair of graspers comprises moving a pivoting portion of the handle relative to a fixed portion of the handle.

32. The method of claim 31, further comprising locking the first pair of graspers into the first position.

33. The method of claim 32, wherein the pivoting portion of the handle defines a ratchet, and locking the first pair of graspers into the first position comprises engaging a pawl into the ratchet to prevent movement of the pivoting portion in at least one direction.

34. The method of claim 33, wherein the first and second positions are between 0 and about 7 cm apart.
